(19) United States
(12) Patent Application Publication
(10) Pub. No.: US 2020/0163956 A1
(43) Pub. Date: May 28, 2020

(54) DIRECT BRAIN ADMINISTRATION OF CHEMOTHERAPEUTICS TO THE CSF FOR PATIENTS WITH PRIMARY AND SECONDARY BRAIN TUMORS

(71) Applicant: Cerebral Therapeutics LLC, Aurora, CO (US)

(72) Inventors: Daniel J. Abrams, Denver, CO (US); Michael S. Canney, Denver, CO (US); Stephen Farr, Orinda, CA (US)

(73) Assignee: Cerebral Therapeutics LLC, Aurora, CO (US)

(21) Appl. No.: 16/329,522

(22) PCT Filed: Sep. 6, 2017
(86) PCT No.: PCT/US17/50217

§ 371 Date: Feb. 28, 2019

Related U.S. Application Data
(60) Provisional application No. 62/383,726, filed on Sep. 6, 2016.

(51) Int. Cl. A61K 31/4745; A61K 9/00

(52) U.S. Cl. CPC A61K 31/4745; A61K 9/0085; A61K 9/0019

(57) ABSTRACT

In some embodiments, a method may include treating brain cancer sensitive to cytotoxic effect. The method may include intraventricularly administering to a subject via a subject's cerebrospinal fluid an effective amount of a pharmaceutical formulation. The pharmaceutical formulation may include at least one chemical compound. In some embodiments, the pharmaceutical formulation may include at least one aqueous diluent. The at least one chemical compound may include a molecular weight of between about 400 MW and about 10,0000 MW. The at least one chemical compound may include protein binding of greater than 30% and greater than 70 Angstroms in cross sectional area. In some embodiments, the at least one chemical compound includes Irinotecan, SN-38, and/or a related derivative thereof. In some embodiments, the method may include ameliorating and/or inhibiting brain cancer in the subject using the pharmaceutical formulation.

### BACKGROUND OF THE INVENTION

### 1. Field of the Invention

The present disclosure generally relates to direct brain administration of a chemotherapeutic agent for patients with primary or secondary brain tumors. More particularly, the disclosure generally relates to methods of use for a group of drug molecules to be delivered directly to the cerebrospinal fluid (CSF) of a patient in order to achieve sufficient effective concentrations in the brain.

### 2. Description of the Relevant Art

### Current Treatment Strategies for Primary or Secondary Brian Tumors

Both primary and secondary brain tumors are primarily treated with a combination of either surgical resection, radiation therapy, and/or systemically administered chemotherapy. Chemotherapeutics are typically administered via intravenous (IV) or oral routes of administration. Radiation therapy can consist of either targeted radiotherapy or whole brain radiation. Surgical therapy primarily involves biopsy for diagnoses and when possible gross tumor removal with margins. These three types of interventions have significant side effects and have relative limited benefit as they only extend survival by a few months.

### Primary Brain Tumors

Malignant parenchymal CNS neoplasms of glial origin (primary brain tumors /gliomas) have some of the worst outcomes in oncology. Approximately 10,000 patients die each year with a median survival of 15 months with progressive neurologic dysfunction occurring as the disease progresses. The major advance in the treatment of gliomas has been an increase in the precision of surgical resection in the last 15 years, in particular with the advent of

MRI coupled with neuronavigation, which has facilitated better delineation of tumor margins during tumor resection. In addition, focused beam radiation coupled with improvements in imaging technology has become more commonly used and in the past using implanted radiation therapy (brachytherapy) also held some promise but is not widely used today. Occasionally, a local 4 week delayed release chemotherapeutic wafer (carmustine implant, Gliadel®) is used as well based on the concept that local delivery of a delayed release implant that contains a drug to the resection cavity is responsible for its very modest effectiveness. Unfortunately, these advancements have extended life expectancies by at best only a few weeks with the natural history of the disease still a quick relentless demise. To emphasize, as mentioned above, a local 4 week delayed release chemotherapeutic wafer (carmustine implant, Gliadel®) has been approved and used as a delayed release implant placed as an implant within the brain in the resection cavity. However, the wafer is widely recognized to be ineffective at achieving sufficient drug levels in order to limit the biologic activity of the residual cancer cells and is a very limited way despite significant unmet need for patients.

Despite, the recent introduction of new drug therapies such as Avastin® (bevacizumab) and devices such as the Optune® system (Novocure) for patients with glioblastoma have been shown to increase quality of life in patients, still there has not been a significant improvement in patient survival. Fifty years ago, the introduction of steroids and radiation treatment added 6 to 10 months to median survival, which was a greater improvement in patient survival than all of the recent treatments combined.

### Secondary Brain Tumors

Malignant metastases to the brain parenchyma are very common, portend short survival, and are most commonly treated with whole brain radiation and sometimes local resection and focused beam radiation. Brain metastases are often the reason patients die from cancers that originate in the breast, skin, lungs, and other organs. The total dose of radiation that can be delivered is limited as patients receiving whole brain radiation can suffer from devastating cognitive deficits. Patients with brain metastases who receive whole brain radiation of approximately of 5000 rads suffer from cognitive deficits if they live for longer than a year. In relative similarity to glioblastoma, the prognosis from metastatic stages of systemic cancers that have metastasized to the brain are very poor.

Significant advances in drug therapies that have emerged for other cancers (e.g. breast, lung, colon, skin) but unfortunately have not benefited patients with brain cancer. Drug therapies approved for primary brain tumors have included the nitrosoureas (BCNU, CCNU) in the 1970s, temozolomide (Temodar®) in the late 90s, and bevacizumab (Avastin®) in the last 10 years. These drug therapies have led to modest increases in a few months in median survival for patients with primary brain tumors. Patients with secondary brain tumors largely do not respond to drug therapies that are effective on tumors originating in other organs and thus radiation is often used to treat secondary brain tumors. There is significant unmet need in primary and secondary brain tumors for improved methods of treatment.

### Blood-Brain Barrier

One of the primary reasons why drug therapies are not effective in the brain is due to the presence of the blood-brain barrier (BBB), which limits the passage of nearly all large molecule and the majority of small molecules (<500 Da) to the brain parenchyma. Due to the presence of the BBB, most drugs that are administered orally or by intravenous infusion do not reach sufficient concentrations in the brain parenchyma to have therapeutic effects. As such potential drugs that may have the highest potential efficacy against cancerous tissue are unable to reach the target tissue due to the BBB. For example, trastuzumab (Herceptin®), a humanized IgG1 kappa monoclonal antibody for the treatment of metastatic breast cancer has a CSF level that is 300-fold lower than plasma levels when administered intravenously.

Several methods have been proposed to bypass the BBB to increase drug concentrations in the brain. These methods include convection-enhanced delivery (CED), intra-arterial injection, osmotic solutions (mannitol) to disrupt the BBB, ultrasound, direct injection to the CSF, high dose systemic chemotherapy, and drug-loaded wafers inserted directly into the tumor resection cavity (Gliadel® wafers).

### CSF Drug Delivery and Leptomeningeal Disease

One method that has been abandoned as ineffective is drug delivery to the cerebrospinal fluid (CSF) for treatment of diseases within the brain. Drug delivery to the CSF is only used for treatment of cancer that is in the lining, leptomeninges or ependyma of the brain and spinal cord. This type of cancer is referred to as leptomeningeal metastases or primary leptomeningeal disease.

Cancer agents administered into the CSF have been widely used to treat leptomeningeal disease but not disease of the brain parenchyma. Such agents administered to treat leptomeningeal disease are typically injected into the CSF through either the intralumbar or the intraventricular routes. It has been widely believed that because of the flow of CSF from the parenchyma towards the ventricles, the direction of equilibrium between the ventricular CSF and the extra cellular space for small molecules administered into the ventricular CSF therapeutic concentrations cannot be reached in the parenchyma. For larger, less diffusible molecules, it has been believed that equilibrium between the two compartments never occurs because diffusion is too slow. This has been described as the CSF-brain barrier and is the presumed reason why intra-CSF drug administration is an inefficient and ineffective delivery strategy for parenchymal tumors. Today CSF chemotherapy is used for leptomeningeal metastases and for CNS prophylaxis for high-risk leukemia. The two drugs that are used most frequently for treatment of leptomeningeal disease are methotrexate and cytarabine.

Direct cerebrospinal fluid infusion of drug therapies for brain diseases, besides for leptomeningeal cancer, has largely been abandoned since the 1970s. In the classic experiments done at the National Institutes of Health, it was concluded that that the convective CSF fluid production makes it such that injected drugs do not reach brain tissue at more than several millimeters in effective concentrations. It should be noted that the five drug molecules (hydroxyurea, methotrexate, thiotepa, BCNU, cytosine arabinoside) tested in these studies had CSF half-lives of less than two hours for all drugs except one methotrexate which has a half-life of four hours.

### Pharmacokinetics of Intraventricular CSF Administration for Leptomeningeal Disease: Lipophilicity, Protein Binding and Molecular Size

For forty years there have been attempts to utilize systemic medications to accumulate sufficiently and be effective enough to successfully treat both primary and secondary CNS neoplasms. Since the BBB is changed or altered in the region of the tumor to what is called the blood tumor barrier (BTB), it was thought that the greater opening to molecule penetration would suffice for chemotherapies to work when administered systemically. Unfortunately, with almost no exception, despite this variation in the brain tumor barrier in the area of the tumor there has not been any significant success using systemic administration of chemotherapeutics.

Please see Table 1 below for a summary of molecules used to treat diseases within the leptomeninges via CSF infusion; but not used for diseases extending beyond the internal brain coverings into the brain substance. To emphasize the state of knowledge today, medications have been useful for diseases not in the brain parenchyma but in the brain coverings. The key item that has been emphasized is CSF pharmacokinetics with brief infusion and the resulting alpha or initial half-life and peak CSF concentration. Specifically, these CSF based therapies have not been used to treat, through CSF ventricular infusion, diseases of the brain parenchyma. Interestingly etoposide is large enough with a high enough molecular weight, and significant protein binding (above 80%), to be considered for central administration to treat parenchymal disease if the dosing approach might be modified. In addition, methotrexate (please see section below on co-administration) may also have some of the same potentials for direct brain administration, though given its substantial CNS toxicity profile, related possibly to cumulative dose accumulation, is likely difficult to overcome even with an altered dosing regimen. Topotecan which is somewhat smaller but perhaps still large enough only has 30% binding so it still may be an option if the right dosing regimen is utilized in the future. Interestingly, intraventricular topotecan was used in a study for meningeal metastases with short-term bursts of administration and was not found to have added benefit. This stands in contrast to molecules such as 5 Flourouracil, which have protein binding of approximately 10% and Ara C, with protein binding under 15%. Both of these are small molecules that are unlikely to be candidates for longer-term direct CSF administration to treat diseases of the brain parenchyma.

TABLE 1
Summary of molecules used to treat diseases within the leptomeninges via CSF infusion.

| | | Molar | | t_{1/2b} | | | | | | | |
|---|---|---|---|---|---|---|---|---|---|---|---|
| Drug | Formula | Mass | t_{1/2a} (hours) | (hours) | Vdss (mL) | AUC_{csf} | | AUCₛₑᵣᵤₘ | | Cmax | |
| Methotrexate | C₂₀H₂₂N₈O₅ | 454 | 1.7 | 6.6 | 70 | | | | | >200 | mmol/L |
| Cytarabine | C₉H₁₃N₃O | 243 | 1 | 3.4 | | 354 | mmol min/L | | | >2 | mmol/L |
| Thiotepa | C₆H₁₂N₃PS | 189 | CSF: 100 ug/mL -> 10 | | | 5470 | mg min/mL | 20 | ug min/mL | >100 | mg/mL |
| | | | ug/mL (2 hours) -> 1 ug /mL | | | | | | | | |
| | | | (8 hours) | | | | | | | | |
| DTC 101 - | C₉H₁₃N₃O₅ | 243 | 9.4 | 141 | | | | | | 75 | mg to |
| liposomal | | | | | | | | | | CSF - | |
| cytarabine | | | | | | | | | | MTD | |
| Mercaptopurine | C₅H₄N₄S | 152 | 1.4 | | | 1941 | mmol h/L | 2.68 | umol h/L | 763 | mmol/L |
| Mafosfamide | C₉H₁₉Cl₂N₂O₅PS₂ | 401 | 0.4 (primate) | | | | | | | 100 | mmol/L |
| Etoposide | C₂₉H₃₂O₁₃ | 589 | 1.14 | 7.41 | 160 | 25.0 | mg h/mL | | | 9.03 | mg/mL |
| Topotecan | C₂₃H₂₃N₃O₅•HCl | 458 | ? | 2.8 | | | | | | 28 | mmol/L |
| Nimustine | C₉H₁₃ClN₆O₂ | 273 | 0.4-0.6 | | 24-101 | 14.7 | mg min/mL | | | 620 | mmol/L |
| (ACNU) | | | | | | | | | | | |

### Potential Reasons Why Systemic Administration Was Not Successful for Irinotecan—Irinotecan was Unable to Achieve a Targeted Therapeutic Dose Through Systemic Delivery Methods

Some CNS pharmacokinetic parameters with systemic CPT-11 administration have been evaluated. In non human primate studies, Blaney et al found a max CSF concentration of 240 nM of CPT-11 after an 11.4 mg/kg IV administration which is equivalent to 225 mg/m² between 30 and 60 minutes after an IV dose. At a therapeutic systemic bolus dose, Blaney et al. found that CSF levels are only 1/40 of low end for IC50 of CPT-11.

### Convection Enhanced Delivery (CED) Delivery for Brain Disease—a Different Approach

A completely different approach from CSF based infusion, called Convection Enhanced Delivery (CED), has been explored extensively; probably in part at least due to the fact that ventricular CSF based drug delivery to the brain parenchyma has been abandoned.

Since the abandonment of CSF infusion for disease of the brain parenchyma, CED, which is direct injection into the brain parenchyma, under pressure and with a specific flow rate, has dominated direct brain drug delivery for the last twenty plus years. CED approaches have focused on brain cancer and Parkinson's Disease but are also now being explored for purpose of treatment of genetic and Alzheimer' s diseases. CED type brain infusion requires volumes of one to two microliters per minute.

To date, while there are some promising therapies for CED in development, there is still substantial unmet need and technical challenges with CED making it questionable whether the therapies will successfully be developed to help patients.

In addition to the location differences between CED and ventricular CSF based administration, there are other important differences between CED and CSF based infusion in terms of volume infused, length of infusion, molecules which are reasonable to be used, volume and target of infusion area, chronicity of infusion and molecular capability and relationship to an external pump. For CED, the infusion rate is relatively fixed with a requirement of ˜1 microliter per minute whereas for CSF based infusion, the amount could be just a few microliters a day to several milliliters a day and is far less restricted. CSF-based infusion and CED both in theory could be used for chronic or long-term infusion, but because of the requirement of a relatively large infusion volume for CED, the ability to have longer-term infusions is practically limited by storage volumes, need for refills, size of pumps etc. CED is intended for a targeted volume of a specific amount around a catheter and can require several catheters to cover a larger area or an area that has a complex geometry whereas CSF based delivery is limited by the potential of the molecule distribution. In theory both CED and CSF based infusion could use any kind of molecule but practically CSF based infusion is limited by the right molecule being able to distribute sufficiently in the target area based on distribution and residence times in the right dosing circumstance, whereas for CED, the area of infusion is the primary limitation without a primary molecular limitation. While both CED and CSF based infusion can be utilized with an implantable or a chronic pump, practically CSF based infusions will be determined by the length of therapy time and CED infusions will less likely be mated with an implantable pump given the diseases and volume used for chronic delivery.

Accordingly, there is a need in the art for a method to deliver drugs to the ventricular cerebrospinal fluid with distribution to the brain parenchyma for both primary and secondary brain tumors that avoids systemic toxicities and that leads to therapeutic concentrations in brain tissue.

### SN-38, an Additional Reason That CSF Based Administration of Irinotecan (CPT-11) is Promising

From the product label, “Over the recommended dose range of 50 to 350 mg/m², the AUC of Irinotecan increases linearly with dose; the AUC of SN-38 increases less than proportionally with the dose. Maximum concentrations of the active metabolite SN-38 are generally seen within 1 hour following the end of a 90-minute infusion of Irinotecan.” From studies surrounding approval of the active metabolite SN-38, some additional key facts should be pointed out, first, the major excretion product was unchanged CPT-11, accounting for 55% of the administered dose, followed by APC (10.5%), SN-38 (8.7%), SN-38G (3.3%), and NPC (1.5%). Second, inter-individual variation in CPT-11 pharmacokinetics was reported in several studies. And third, major dose-limiting toxicities of CPT-11 therapy are diarrhea and leukopenia from systemic administration. There is an equilibrium of Irinotecan and SN-38 between the lactone and hydroxyl acid form which may facilitate appropriate binding for toxicity and availability to attack target DNA with the ideal toxicity being of the lactone form with a lower pH.

Interestingly, there is variation in potency of SN38 and Irinotecan while the concentration of Irinotecan is much higher than the SN-38 concentration. From the label: In vitro cytotoxicity assays show that the potency of SN-38 relative to Irinotecan varies from 2- to 2000-fold; however, the plasma area under the concentration versus time curve (AUC) values for SN-38 are 2% to 8% of Irinotecan and SN-38 is 95% bound to plasma proteins compared to approximately 50% bound to plasma proteins for Irinotecan.

Accordingly, there is a desire to provide a device for the direct administration of drug therapies to the brain through an intraventricular access device that allows for access to the CSF through a port located just beneath the scalp of a patient.

### SUMMARY

In some embodiments, a method may include treating brain cancer sensitive to cytotoxic effect. The method may include intraventricularly administering to a subject via a subject's cerebrospinal fluid an effective amount of a pharmaceutical formulation. The pharmaceutical formulation may include at least one chemical compound. In some embodiments, the pharmaceutical formulation may include at least one aqueous diluent. The at least one chemical compound may include a molecular weight of between about 400 MW and about 10,0000 MW. The at least one chemical compound may include protein binding of greater than 30% and greater than 70 Angstroms in cross sectional area. In some embodiments, the at least one chemical compound includes Irinotecan, SN-38, and/or a related derivative thereof. In some embodiments, the method may include ameliorating and/or inhibiting brain cancer in the subject using the pharmaceutical formulation.

In some embodiments, the pharmaceutical formulation is administered for periods of longer than about 8 hours at a time. The pharmaceutical formulation may be administered not less than every four weeks at least during the initial few months of administration.

In some embodiments, the method may include solubilizing the at least one chemical compound in the at least one aqueous diluent. The chemical compound may be solubilized using pegylation, liposomal encapsulation, emulsion carrying system, microgrinding into nano particles, or cyclodextrins.

In some embodiments, the chemical compound may include a pharmaceutically acceptable salt thereof. The chemical compound may include Irinotecan, SN-38, and/or a related derivative thereof. The at least one chemical compound may include Irinotecan, wherein the method further comprises administering Irinotecan at about 5 to about 200 mgs per day for a period of administration of not fewer than 8 hours. The at least one aqueous diluent may include 5% Dextrose or at 0.9% Sodium Chloride. In some embodiments, the method may include administering Irinotecan such that SN-38 achieves levels above 18.0 pmol of SN-38 and possibly higher than SN-38 IC₅₀=10-750 nm (low end=U251, high end=U87) CPT-11 IC₅₀=10 uM-85 uM (low end=U251, high end=U87) will be sampled via an implantable CSF sampling device and via intermittent brain biopsies.

Additional oncology drugs may exhibit behavior similar to CPT-11 and SN-38 and their derivatives because of their size and protein binding. In some embodiments, the chemical compound may include abraxane, Cabazitaxel, carfilozimb, docetaxel, doxorubicin, Etirinotecan pegol (NKTR-02), etoposide, NKTR-105, omacetaxine mepesuccinate, topotecan, paclitaxel, lapatinib, temsirolimus, or trametinib. In addition, molecules that can be made to be larger via covalent processes like pegylation achieve the same result and have the same characterization.

In some embodiments, the method may include administering to the subject a pharmaceutical manufactured form of carboxylesterase inducing further conversion of CPT-11 to

SN-38 (e.g., as depicted in FIGS. 1-2) and to expand the bioavailability of SN-38 to further treat the brain cancer.

In some embodiments, the method may include administering to the subject a pharmaceutical manufactured form of atropine could be co-administered centrally to further tolerance of the medication. As another illustrative example, oral methylphenidate can be used for asthenia, at a dose around 10 mg twice a day. As another illustrative example, steroids (e.g. decadron 4 mg IV or 0.1 mg ICV) could be administered to diminish cases of aseptic ventriculitis potentially caused by irritation from central drug infusion.

In some embodiments, the method may include adjusting a concentration of the at least one chemical compound based upon sampling of the subject's cerebrospinal fluid.

In some embodiments, the method may include administering the pharmaceutical formulation to the subject via a treatment course which lasts at least two weeks and extends indefinitely.

In some embodiments, the brain cancer comprises metastatic cancer including small cell lung cancer, gastrointestinal cancer, breast cancer, testicular cancer, pancreatic cancer.

Primary brain tumors may include glioblastoma, anaplastic astrocytoma and glioma.

In some embodiments, the method may include administering the pharmaceutical formulation via a long catheter that is connected to either an implantable pump or an externalized pump for greater than 12 inches of catheter under the skin and preferably longer. In some embodiments, the method may include administering the pharmaceutical formulation using a kit including an implantable pump system including separately or together a ventricular catheter, an infusion catheter, a sterility packaging, patient identification card, infusion system identification card.

In some embodiments, the method may include administering the pharmaceutical formulation to a thecal space of the subject depending on their toxicity profile.

### BRIEF DESCRIPTION OF THE DRAWINGS

Advantages of the present invention may become apparent to those skilled in the art with the benefit of the following detailed description of the preferred embodiments and upon reference to the accompanying drawings.

FIG. 1 depicts a diagram of Irinotecan (CPT-11) administered to the ventricular CSF of a subject diffusing through the brain parenchyma to the tumor, where it is converted to SN-38 by enzymes in the tumor region. SN-38 is highly protein bound and remains in the region of the tumor, where it is cytotoxic and kills the tumor.

FIG. 2 depicts a diagram of the molecule CPT-11 and its metabolite SN-38.

FIG. 3 depicts a diagram of an implantable drug pump implanted near the abdomen and drug delivery is performed directly to the CSF of a subject via an implanted catheter in the lateral ventricles.

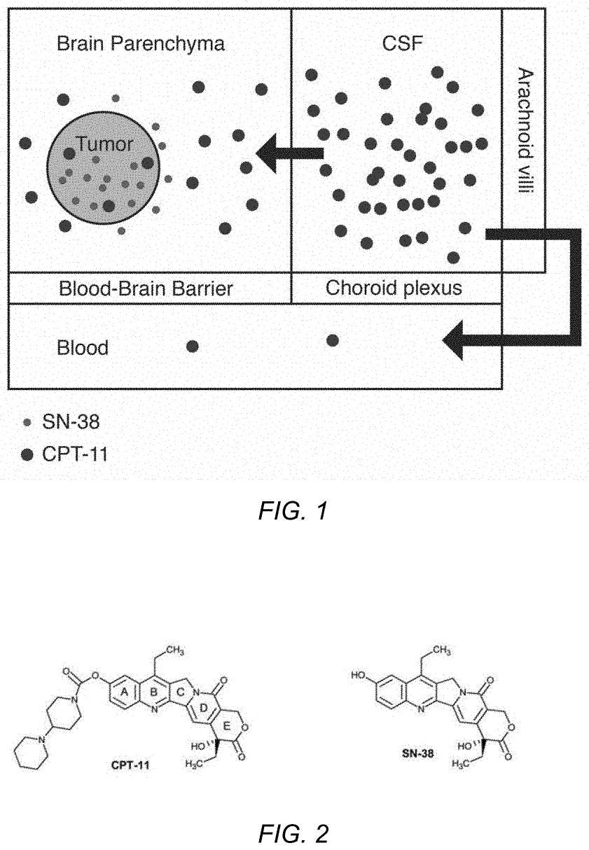

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must). The words “include,” “including,” and “includes” indicate open-ended relationships and therefore mean including, but not limited to. Similarly, the words “have,” “having,” and “has” also indicated open-ended relationships, and thus mean having, but not limited to. The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “third die electrically connected to the module substrate” does not preclude scenarios in which a “fourth die electrically connected to the module substrate” is connected prior to the third die, unless otherwise specified. Similarly, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a set of electrical conductors may be configured to electrically connect a module to another module, even when the two modules are not connected). In some contexts, “configured to” may be a broad recitation of structure generally meaning “having circuitry that” performs the task or tasks during operation. As such, the component can be configured to perform the task even when the component is not currently on. In general, the circuitry that forms the structure corresponding to “configured to” may include hardware circuits.

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component.

The scope of the present disclosure includes any feature or combination of features disclosed herein (either explicitly or implicitly), or any generalization thereof, whether or not it mitigates any or all of the problems addressed herein. Accordingly, new claims may be formulated during prosecution of this application (or an application claiming priority thereto) to any such combination of features. In particular, with reference to the appended claims, features from dependent claims may be combined with those of the independent claims and features from respective independent claims may be combined in any appropriate manner and not merely in the specific combinations enumerated in the appended claims.

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a linker” includes one or more linkers.

### DETAILED DESCRIPTION

### Definitions

Unless defined otherwise, all technical and scientific terms used herein have the same meaning as commonly understood by one of ordinary skill in the art.

The term “catheter” as used herein generally refers to medical devices that can be inserted in the body to treat diseases or perform a surgical procedure.

The term “connected” as used herein generally refers to pieces which may be joined or linked together.

The term “coupled” as used herein generally refers to pieces which may be used operatively with each other, or joined or linked together, with or without one or more intervening members.

The term “directly” as used herein generally refers to one structure in physical contact with another structure, or, when used in reference to a procedure, means that one process effects another process or structure without the involvement of an intermediate step or component.

### Overview of Ideal Molecular Properties—Solubility, Molecular Weight and Protein Binding

Herein a class of molecules are defined that have not been previously characterized that will be successful for direct ventricular brain fluid administration resulting in longer CSF half-lives and longer intraparenchymal brain persistence when administered into the ventricular CSF. The longer persistence and time of de facto exposure to the tumor of these molecules from the CSF will allow them to achieve therapeutic doses in the brain parenchyma when administered with the described dosing regimen. .

In some embodiments, the direct administration to the CSF of a certain class of molecules will provide a novel approach to extend life for patients with primary and/or secondary brain tumors. In some embodiments, chronic administration of these molecules could also potentially work when administered within the brain parenchyma if they have the features of a larger molecular weight, segregation to stay within the brain side of the blood brain barrier and enhanced protein binding compared with typical small molecule chemotherapeutics when administered with the described dosing regimen.

By extending the half-life of brain drugs and ideally coupling the drug delivery to pumps (implantable and not), tumor suppression and survival may be significantly improved. In particular, if the drug is properly chosen in terms of its toxicity, molecular weight, protein binding, and degradation product, it will be effective in treating brain tumors through direct CSF administration.

### Effective Therapy for Brain Drug Infusion

In some embodiments, chemical compounds and methods of treatment described herein are different than have been used before, such that the brain residence time will be sufficient for effectiveness. Molecules described herein may have a CSF half-life and brain penetration that is vastly improved in comparison to the previous molecules that have been studied.

### Irinotecan

In some embodiments, a chemical compound may include Irinotecan (CPT-11). Irinotecan includes a molecular weight of greater than 450 daltons, polar surface area greater than 80 (approximately 113 Angstroms) and protein binding to Albumin 30-60% which may combine to give it the ability stay present in the CSF circulation for a much longer duration than other molecules. Irinotecan has substantial toxicity when administered systemically (including hematological cell toxicity, diarrhea), which can be potentially minimized by lower total doses by direct injection into the CSF. In addition, peak drops in blood counts when Irinotecan is administered systemically occurs after 10 days, which with a lower cumulative dose and lower peak dose, may result in peak blood drops becoming less prominent. In addition, the current systemic side effects happen during the usual 90 minute systemic infusion on a weekly basis or less. Lower total dosing and longer infusion cycles may well help to minimize systemic side effects.

Irinotecan is a topoisomerase I inhibitor that is currently approved for the treatment of metastatic colorectal cancer (i.e., Camptosar®, irinotecan hydrochloride injection). Camptosar®is approved for systemic administration during a 90-min infusion in either a) a weekly regimen or b) as a once-every-3-week regimen. For the weekly regimen, the dose is 125 mg/m² and the drug is administered lx per week for 4 weeks, followed by a 2-week rest period. For the once-every-3-week regimen, the drug is administered at a dose of 350 mg/m². The patient may continue additional cycles of drug therapy as long as they continue to experience clinical benefit.

Irinotecan is also approved in a nanoliposomal form (Onivyde®, irinotecan liposomal injection, Merrimack Pharmaceuticals) for the treatment of metastatic adenocarcinoma of the pancreas. Onivyde® is administered at 70 mg/m² by intraveneous infusion over 90 minutes every 2 weeks.

### Conversion of CPT-11 to SN-38

Irinotecan is additionally particularly well suited as an agent for direct administration in the CSF because its metabolite SN-38 is 1000 more potent an oncoloytic, is strongly (>90%) protein bound and SN-38 is not water soluble. In addition, though SN-38 has a smaller molecular weight than 450 it is heavily protein bound and has a relatively large polar surface area of greater than 80 (calculated at 99.96 Angstroms) keeping it preferentially within the CSF space.

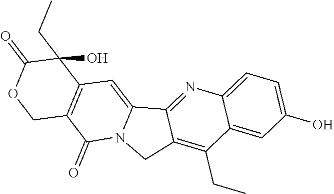 [SMILES: CCc1c2c(nc3ccc(O)cc13)-c1cc3c(c(=O)n1C2)COC(=O)[C@]3(O)CC]

Transformation of Irinotecan into SN-38 occurs via carboxylesterases. Conversion of Irinotecan to SN-38 is usually performed by carboxylesterases which reside in the liver with systemic administration; but recent studies have shown that the drug is also converted to SN-38 by enzymes in glioma cells.

### Pharmacodynamics of Irinotecan (CPT-11)

In some embodiments, SN-38 is approximately 1000 times as potent as Irinotecan as an inhibitor of topoisomerase I purified from human and rodent tumor cell lines. In vitro cytotoxicity assays show that the potency of SN-38 relative to Irinotecan varies from 2- to 2000-fold. The precise contribution of SN-38 to the activity of Irinotecan Hydrochloride Injection, USP is thus unknown.

Therefore, direct injection of Irinotecan into the ventricular CSF and subsequent distribution to cancerous cells will lead to both higher local concentrations of Irinotecan as well as local conversion to SN-38 in the targeted region of cancer and adjacent to cancer areas. In some embodiments, a pharmaceutically manufactured form of carboxylesterase could be co-administered centrally to supplement conversion of Irinotecan to SN-38 and to expand the bioavailability of SN-38 to further treat the tumor cells. Irinotecan has shown activity against colorectal cancer, pancreatic and ovarian cancer systemically and would be expected to show that activity also in the central nervous system when administered on the brain side of the blood brain barrier for treatment of systemic tumors which have progressed to brain metastases. Both Irinotecan and SN-38 exist in an active lactone form and an inactive hydroxyl acid anion form which are in rapid dynamic equilibrium. SN-38 is not a P-glycoprotein substrate, and its cytotoxicity toward tumor cells is not notably diminished by multidrug-resistance overexpression.

The fraction of SN-38 bound to plasma proteins is very high (92-96%) in comparison to Irinotecan (30-43%). When Irinotecan is converted to SN-38, the molecule is both highly cytotoxic and highly protein-bound, therefore, it will preferentially accumulate near the tumor and not be rapidly cleared from the CSF and brain parenchyma. In some embodiments, SN-38 may have a serum half-life of 11 hours or longer and a sufficiently long CSF and brain based residence time at a sufficient concentration to achieve an oncolytic effect.

### Other Potential Drugs for Parenchymal Disease Treatment via CSF Administration Include

Additional oncology drugs that may exhibit similar behavior to Irinotecan and SN-38 and their derivatives because of their size and protein binding. In some embodiments, chemical compounds may include: abraxane, Cabazitaxel, carfilozimb, docetaxel, doxorubicin, etoposide, omacetaxine mepesuccinate, topotecan, paclitaxel, lapatinib, temsirolimus, or trametinib. These medications have larger molecular weights and preferentially may have extended residence time. A number of these molecules are poorly soluble and for direct brain fluid administration would need be re-formulated via a liposomal carrier, converted into very small particles or albumin microparticles, or by other methods of making liquid administration with poorly soluble drugs (cyclodextrins etc.). In the Non-Oncology settings other drugs could also potentially be explored with this profile.

### Co-Administration Centrally or Peripherally, With Irinotecan, Other Medications to Address a Side Effect or Toxicity of the CSF Administered Agent

Irinotecan administered centrally may or may not elicit a cholinergic syndrome which may in part depend on the dosing strategy. In some embodiments, atropine may be co-administered peripherally or centrally depending on the cholinergic pattern that emerges. Similarly, Irinotecan patients can develop an asthenia syndrome which could be treated with methylphenidate.

In some embodiments, using herein described method treatment systems one could administer locally and centrally inhibitors of the drug transporters. As an example, for etoposide, for the p-glycoprotein (HUGO name ACBC1) and inhibitors like verapamil, cyclosporine A, or quinidine or if the transporter is MRP 6 (Hugo name ABCC 6) inhibitors like probenenecid and indomethacin may be used. Even though drug-drug interactions related to the liver are less of an issue with central administration, care still needs to be taken because of brain transporter inhibition.

In some embodiments, Irinotecan may be administered with other chemotherapies to enhance synergistic effect so long as that effective concentrations of complementary acting medications. The complementary medications, could be centrally administered or one centrally and others peripherally if they are effective peripherally.

### Differing Dosing Schedules and “Dose Intensity”

There are several differing dosing strategies that may take advantage of the uniqueness of CSF central dosing for patients with glioblastoma to maximize therapeutic efficacy and maximize tolerability. In some embodiments, Irinotecan may be administered a week on therapy and a week off of therapy; the option highlighted below. Patients may be started at 5 mg, then 10 mgs a day, kinetics will be obtained, and CSF levels will be determined. Once the residence time is determined a dose escalation may proceed until a maximum tolerated dose is established. The dose may be increased to 20 mgs per day and then raised by 20 mgs each dose until 80 mgs a day are reached. In between each dose level, one week of therapy at each level, the pump may be turned off for a week as each level is increased. The treatment escalation may be stopped at 80 mgs or alternatively earlier at a dose half way between a toxic dose and the next lower dose. After the maximum tolerated dose is determined the patients may continue for a total of 6 months or longer of treatment. A key of dosing is toxicology and tolerability rather than synchronization to the cell cycle, and essentially tolerability.

In some embodiments, differing dosing regimens, each involving a dose escalation scheme for establishing the dosing range of either a maximum tolerated dose or maximum infusible dose, may include:

1) Constant infusion dosing;
2) Infusions for 5 half-lives and continuing for a week beyond that (half-lives to be determined by PK studies);
3) One week on and one-week treatment off—concept of “prolonged cycles” may be two weeks on and two weeks off or some other phase that is longer than 90 minutes or 4 hours;
4) Longer term treatment than a limited number of cycles such that continued repression can be utilized so over many months;
5) Infusion targeted by terminal half-life and residence time above targeted level from prior in vitro level or through personalized patient experimentation based on determined sensitivity. Personalized medication variant possibly tied to genotyping and in vitro testing;
6) New Levels established by Maximum Tolerated Doses or Surrogate mapping; and
7) Addition of a second agent at either the same time, alternating, with time on or off or concurrently.
8) If using a longer acting formulation intracerebroventricularly, dosing can be shorter and/or intermittent because of the extension of the half life through such methods as liposomal encapsulation or pegylation amongst other

| | Around the clock - | Around the clock - cycles. | | One, twice or Three |
|---|---|---|---|---|
| | constant 24/7 | One week one and one | 90 minutes once every | times a day because |
| | except bathroom | week off - how many cycles? | three weeks and lower | of like bolus dosing, |
| | or walking. For | Depending on PK but could extend | to every two weeks | this could be done |
| | how long | this to two weeks on and off | and every week | with CED |
| Other | Can add systemic | Can add another | Can add another | Can add another |
| Notes | | central or | central or | central or |
| | | systemic | systemic | systemic |

### Arguments for Longer Term Infusion Being Beneficial as Opposed to Brief Intermittent Boluses

The current regimen of medications for most systemic chemotherapies is brief and intermittent. An example is that Irinotecan is infused at 90-minute infusion every two weeks or some variations thereof. There has been limited exploration of other infusion regimens which have likely been limited historically by precedent, precedent with the efficacy of chemotherapy cycles clinically and other technical challenges like the availability of pumps or formulation challenges with medications. In addition, the dosing regimes for leptomeningeal treatment, all are relatively short term and intermittent compared with more usual systemic medication treatment.

More frequent and/or persistent infusion to the CSF of drugs may have several advantageous effects. First by extending the time of distribution and making the infusion longer term may result in enhancing the ability to have an increased median residence time to facilitate greater effective distribution of the chemotherapy throughout the brain and the tumor. As total dose is likely less than when systemically administered, systemic toxicity related to cumulative dose and peak systemic dose (idiosyncratic toxicity would not necessarily be impacted) may be greatly reduced.

Furthermore, the therapeutic level of drug needed with constant infusion may be much lower than that needed when the drug is administered at discrete time points and this may impact cumulative doses. For example, it was found that the dose of topotecan needed to induce the same cytotoxic effects was 3-5× lower in vitro in cell culture when cells were exposed for continuous exposure versus only exposing cells for 4 hours out of 24 hours. Lower drug concentrations required with continuous exposure clinically may mean that continuously infusing molecules chronically is desirable, especially since the drugs may be directly injected into the CSF.

Interestingly, it is noted that with the wrong dosing regimen today even with the right drug, it is unlikely to get a therapeutic effect. Longer acting agents including those in liposomes and PEGylated versions applied on the inside of the BBB is another counterintuitive way to retain medications within the CSF and the Central Nervous System for longer.

### After Establishing Toxicity Dose, Establishing Effective Dose With CSF and Brain Sampling

One of the things unique in direct CSF administration is the importance of identifying target dosages and using those to monitor drug levels. Currently the approach primarily focuses on half-life or alpha half-life as opposed to dose at steady state where a steady state is established. Because of the unique nature of drug delivery into the Cerebrospinal fluid clinical toxicology and dose level tolerance is key but in terms of effectiveness and monitoring between patients direct (tumor or brain parenchyma) or indirect (cerebrospinal fluid) measures of drug level are critical. Once alpha and beta half-life are determined and a time to establish effective concentration are established during a maximal tolerated dose, establishing effective concentrations and doses are key. The in vitro background to this will be in part built upon the levels established in Wang et al. 2011. The methods used to identify levels for Irinotcan will be an adaptation of the methods from Metz et al. 2013 Methods for SN-38 and Irinotecan Assays.

### Formulation

Irinotecan reconstituted form may be used in direct infusion. The concentration of the medicine may be approximately 2 mg/ml. The formulation is stable an may be administered in standard diluents of normal saline or water to the CSF.

### Anticipating and Strategy to Address a Potential Side Effect

From the label: “During intravenous administration of CPT-11, typically at doses of 50 to 350 mg/m2, a cholinergic syndrome is frequently observed (Gandia et al., 1993; Abigerges et al., 1995; Bugat et al., 1995; Petit et al., 1997). This includes rhinitis, increased salivation, miosis, lacrimation, diaphoresis, flushing, and intestinal hyperperistalsis. These symptoms can be rapidly alleviated by atropine, suggesting that the side effects result from interaction of the parent drug with acetylcholinesterase (AChE).” Irinotecan administered centrally may or may not elicit a cholinergic syndrome which may in part depend on the dosing strategy and whether the cholinergic syndrome is centrally or peripherally mediated. In some embodiments, additional chemical agents or compounds may be co-administered to ameliorates some of the side effects of the primary chemical compound (e.g., Irinotecan). Atropine in theory may be co-administered peripherally or centrally depending on the cholinergic pattern that emerges. The current protocol for Irinotecan anticholinergic syndrome is atropine 0.2 mg subcutaneous prior to infusion, maybe get used to it easier with increased dosing. Similarly, should asthenia become an issue systemic methylphenidate may be used to mitigate this potential side effect. Sometimes agents may cause an aseptic meningitis due to an irritation of the agent on the CSF. In some embodiments co-administration with steroids may prophylactically address this issue as potentially would a lower and slower administration regime.

### Method of Infusion via a Long Term Catheter and a Pump

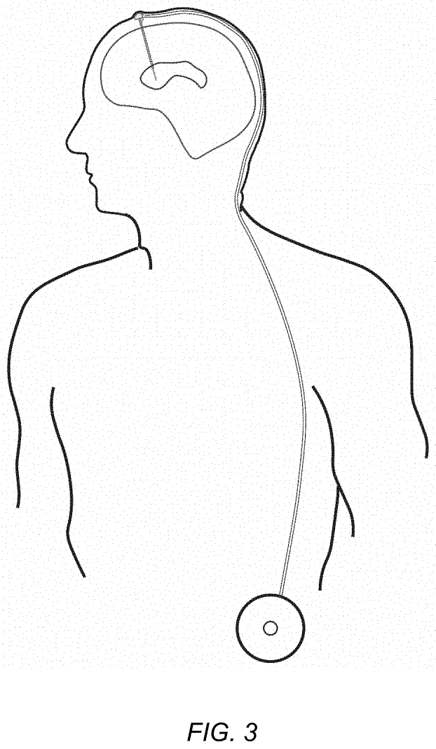

Administration to the CSF via the ventricles or tumor resection cavity may be accomplished via a catheter implanted into a CSF area of collection like the lateral ventricle or the resection bed. Using such catheters, continuous or repeated injections at differing intervals may be performed with drugs into the CSF circulating in and around the brain. In some embodiments, an external syringe pump or alternately an implantable drug pump (e.g., Prometra from Flowonix, or SynchroMed II from Medtronic) may be used to facilitate continuous infusion of drugs or may be used to administer drugs on differing schedules but more frequent than infrequent intermittent boluses (see the section on dosing schedules) (e.g., as depicted in FIG. 3).

### Summary

Today the common wisdom is that brain fluid drug delivery does not work as a primary or adjunct for cancer within the brain substance and only works for ependymal cancer. However, disclosed embodiments herein have found that brain fluid drug delivery is viable for intraparenchymal delivery if you have the right molecule, administered in the right dosage and with the right schedule. The brain may be a selective smaller compartment and, for primary brain tumors at least, allows concentration of higher doses of medication within the neural axis and limiting exposure outside to vulnerable normal dividing cells outside the neural axis. There is a functional selection between three groups of cells including brain cancer GBM stem or progenitor cells which divide a lot, neuraxis cells that typically divide infrequently, and sensitive normal dividing cells outside the neuraxis. Central brain fluid administration targeting dividing cells within the neuraxis having higher levels of exposure makes sense. Essentially the neuraxis fluid delivery can act, for the right molecule, as a compartment to keep tumor toxic agents exposed to the right cells. In some embodiments, an implantable catheter may facilitate dosing flexibility. In some embodiments, the neuraxis is a relatively well vascularized soft tissue which should facilitate drug perfusion during the period when there are living targeted cancerous cells.

CLAUSE A: A pharmaceutical formulation comprising at least one chemical compound and at least one aqueous diluent, for ameliorating and/or inhibiting brain cancer sensitive to cytotoxic effect, wherein the at least one chemical compound comprises a molecular weight of between about 400 MW and about 10,0000 MW, with protein binding of greater than 30% and greater than 70 Angstroms in cross sectional area.

The pharmaceutical formulation of CLAUSE A, wherein the pharmaceutical formulation is administered for periods of longer than about 8 hours at a time.

The pharmaceutical formulation of CLAUSE A, wherein the pharmaceutical formulation is administered not less than every four weeks at least during the initial few months of administration.

The pharmaceutical formulation of CLAUSE A, wherein the at least one chemical compound is solubilized in the at least one aqueous diluent.

The pharmaceutical formulation of CLAUSE A, wherein the at least one chemical compound is solubilized in the at least one aqueous diluent using pegylation, liposomal encapsulation, emulsion carrying system, microgrinding into nano particles, or cyclodextrins.

The pharmaceutical formulation of CLAUSE A, wherein the at least one chemical compound comprises a pharmaceutically acceptable salt thereof.

The pharmaceutical formulation of CLAUSE A, wherein the at least one chemical compound comprises Irinotecan, SN-38, and/or a related derivative thereof.

The pharmaceutical formulation of CLAUSE A, wherein the at least one chemical compound comprises Irinotecan, wherein the method further comprises administering Irinotecan at about 5 to about 200 mgs per day for a period of administration of not fewer than 8 hours, and wherein the at least one aqueous diluent comprises 5% Dextrose or at 0.9% Sodium Chloride.

The pharmaceutical formulation of CLAUSE A, wherein the at least one chemical compound comprises Irinotecan, wherein the method further comprises administering Irinotecan such that SN-38 achieves levels above 18.0 pmol of SN-38 and possibly higher than SN-38 IC₅₀=10-750 nm (low end=U251, high end=U87) CPT-11 IC₅₀=10 uM-85 uM (low end=U251, high end=U87) will be sampled via an implantable CSF sampling device and via intermittent brain biopsies.

The pharmaceutical formulation of CLAUSE A, wherein the at least one chemical compound comprises Etirinotecan pegol, wherein the method further comprises administering Etirinotecan pegol at about 0.5 to 200 mgs per day for a period of administration of not fewer than 60 minutes, and wherein the at least one aqueous diluent comprises 5% Dextrose or at 0.9% Sodium Chloride.

The pharmaceutical formulation of CLAUSE A, wherein the at least one chemical compound comprises Etirinotecan pegol, wherein the method further comprises administering

Etirinotecan pegol such that SN-38 achieves levels above 18.0 pmol of SN-38 and possibly higher than SN-38 IC₅₀=10-750 nm (low end=U251, high end=U87) CPT-11 IC₅₀=10 uM-85 uM (low end=U251, high end=U87) will be sampled via an implantable CSF sampling device and via intermittent brain biopsies.

The pharmaceutical formulation of CLAUSE A, wherein the at least one chemical compound comprises abraxane, Cabazitaxel, carfilozimb, docetaxel, doxorubicin, Etirinotecan pegol (NKTR-02), etoposide, NKTR-105, omacetaxine mepesuccinate, topotecan, paclitaxel, lapatinib, temsirolimus, or trametinib.

The pharmaceutical formulation of CLAUSE A, further comprising a pharmaceutical manufactured form of carboxylesterase inducing further conversion of CPT-11 to SN-38 and to expand the bioavailability of SN-38 to further treat the brain cancer.

The pharmaceutical formulation of CLAUSE A, further comprising a pharmaceutical manufactured form of atropine could be co-administered centrally to further tolerance of the medication.

The pharmaceutical formulation of CLAUSE A, wherein a concentration of the at least one chemical compound is adjusted based upon sampling of the subject's cerebrospinal fluid.

The pharmaceutical formulation of CLAUSE A, wherein the pharmaceutical formulation is administered to the subject via a treatment course which lasts at least two weeks and extends indefinitely.

The pharmaceutical formulation of CLAUSE A, wherein the brain cancer comprises metastatic cancer including small cell lung cancer, gastrointestinal cancer, breast cancer, testicular cancer, pancreatic cancer or primary brain tumors comprising glioblastoma, anaplastic astrocytoma or glioma.

The pharmaceutical formulation of CLAUSE A, wherein the pharmaceutical formulation is administered via a long catheter that is connected to either an implantable pump or an externalized pump for greater than 12 inches of catheter under the skin and preferably longer.

The pharmaceutical formulation of CLAUSE A, wherein the pharmaceutical formulation is administered using a kit including an implantable pump system including separately or together a ventricular catheter, an infusion catheter, a sterility packaging, patient identification card, infusion system identification card.

The pharmaceutical formulation of CLAUSE A, wherein the pharmaceutical formulation is administered to a thecal space of the subject depending on their toxicity profile.

### REFERENCES

The following references are incorporated in their entirety herein:

Abrey, L. E., Chamberlain, M. C., and Engelhard, H., editors (2005). Leptomeningeal Metastases. Springer;
Blaney, S. M., Takimoto, C., Murry, D. J., Kuttesch, N., McCully, C., Cole, D. E., God-win, K., and Balis, F. M. (1998). Plasma and cerebrospinal fluid pharmacokinetics of 9-aminocamptothecin (9-ac), irinotecan (CPT-11), and SN-38 in nonhuman primates. Cancer Chemother Pharmacol, 41(6): 464-8;
Blasberg, R. G., Patlak, C., and Fenstermacher, J. D. (1975). Intrathecal chemotherapy: brain tissue profiles after ventriculocisternal perfusion. J Pharmacol Exp Ther, 195(1): 73-83;
Blasberg, R. G., Patlak, C. S., and Shapiro, W. R. (1977). Distribution of methotrexate in the cerebrospinal fluid and brain after intraventricular administration. Cancer Treat Rep, 61(4): 633-41;
Blasberg, R. G. (1977). Methotrexate, cytosine arabinoside, and BCNU concentration in brain after ventriculocisternal perfusion. Cancer Treat Rep, 61(4): 625-31;
Bobo, R. H., Laske, D. W., Akbasak, A., Morrison, P. F., Dedrick, R. L., and Oldfield, E. H. (1994). Convection-enhanced delivery of macromolecules in the brain. Proceedings of the National Academy of Sciences, 91(6): 2076-2080;
Bomgaars, L., Chamberlin, M., Poplack, D., and Blaney, M. (2002). Leptomeningeal metastases. Cancer in the nervous system, pages 375-390;
Chen, T. C., Su, S., Fry, D., and Liebes, L. (2003). Combination therapy with irinotecan and protein kinase c inhibitors in malignant glioma. Cancer, 97(9 Suppl): 2363-73;
DeVita, V. T., Lawrence, T. S., and Rosenberg, S. A., editors (2008). DeVita, Hell- man, and Rosenberg's Cancer: Principles and Practices of Oncology, Volume 2. Lippincott Williams and Wilkins;
Fleischhack, G., Jaehde, U., and Bode, U. (2005). Pharmacokinetics following intraventricular administration of chemotherapy in patients with neoplastic meningitis. Clin Pharmacokinet, 44(1): 1-31;
Gabay, M. P., Thakkar, J. P., Stachnik, J. M., Woelich, S. K., and Villano, J. L. (2012). Intra-CSF administration of chemotherapy medications. Cancer Chemother Pharmacol, 70(1): 1-15;
Glantz, M. J., Van Horn, A., Fisher, R., and Chamberlain, M. C. (2010). Route of intracerebrospinal fluid chemotherapy administration and efficacy of therapy in neoplastic meningitis. Cancer, 116(8): 1947-52;
Kak, M., Nanda, R., Ramsdale, E. E., and Lukas, R. V. (2015). Treatment of leptomeningeal carcinomatosis: current challenges and future opportunities. J Clin Neurosci, 22(4): 632-7;
Pardridge, W. M. (2012). Drug transport across the blood-brain barrier. J Cereb Blood Flow Metab, 32(11): 1959-72;
Sanghani, S. P., Quinney, S. K., Fredenburg, T. B., Davis, W. I., Murry, D. J., and Bosron, W. F. (2004). Hydrolysis of irinotecan and its oxidative metabolites, 7-ethyl-10-[4-n-(5-aminopentanoic acid)-1-piperidine] carbonyloxycamptothecin and 7-ethyl-10-[4-(1-piperidino)-1-amino]-carbonyloxycamptothecin, by human carboxylesterases ces1a1, ces2, and a newly expressed car-boxylesterase isoenzyme, ces3. Drug Metab Dispos, 32(5): 505-11;
Stein A, Chemotherapy-induced diarrhea: pathophysiology, frequency and guideline-based management Ther Adv Med Oncol. 2010 Jan; 2(1): 51-63;
Stupp, R., Mason, W. P., van den Bent, M. J., Weller, M., Fisher, B., Taphoorn, M. J. B., Belanger, K., Brandes, A. A., Marosi, C., Bogdahn, U., Curschmann, J , Janzer, R. C., Ludwin, S. K., Gorlia, T., Allgeier, A., Lacombe, D., Cairncross, J. G., Eisenhauer, E., Mirimanoff, R. O., European Organisation for Research and Treatment of Cancer Brain Tumor and Radiotherapy Groups, and National Cancer Institute of Canada Clinical Trials Group (2005). Radiotherapy plus concomitant and adjuvant temozolomide for glioblastoma. N Engl J Med, 352(10): 987-96;
Stupp, R., Wong, E T , Kanner, A. A., Steinberg, D., Engelhard, H., Heidecke, V., Kirson, E. D., Taillibert, S., Liebermann, F., Dbaly, V., Ram, Z., Villano, J. L., Rainov, N., Weinberg, U., Schiff, D., Kunschner, L., Raizer, J., Honnorat, J., Sloan, A., Malkin, M., Landolfi, J. C., Payer, F., Mehdorn, M., Weil, R. J., Pannullo, S. C., Westphal, M., Smrcka, M., Chin, L., Kostron, H., Hofer, S., Bruce, J., Cosgrove, R., Paleologous, N., Palti, Y., and Gutin, P. H. (2012). Novottf-100a versus physician's choice chemotherapy in recurrent glioblastoma: a randomised phase iii trial of a novel treatment modality. Eur J Cancer, 48(14): 2192-202;
Stupp R, Taillibert S, Kanner A A, et al. Maintenance Therapy With Tumor-Treating Fields Plus Temozolomide vs Temozolomide Alone for Glioblastoma: A Randomized Clinical Trial. JAMA. 2015; 314(23): 2535-2543. doi: 10.1001/jama.2015.16669;
Wang, W., Ghandi, A., Liebes, L., Louie, S. G., Hofman, F. M., Schönthal, A. H., and Chen, T. C. (2011). Effective conversion of irinotecan to SN-38 after intratumoral drug delivery to an intracranial murine glioma model in vivo. Laboratory investigation. J Neurosurg, 114(3): 689-694;
Wolff, J. E., Trilling, T., Molenkamp, G., Egeler, R. M., and Jurgens, H. (1999). Chemosensitivity of glioma cells in vitro: a meta analysis. J Cancer Res Clin Oncol, 125(8-9): 481-6; and
Wu, M. H., Yan, B., Humerickhouse, R., and Dolan, M. E. (2002). Irinotecan activation by human carboxylesterases in colorectal adenocarcinoma cells. Clin Cancer Res, 8(8): 2696-700.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A method of treating brain cancer sensitive to cytotoxic effects, comprising:
intraventricularly administering to a subject via a subject's cerebrospinal fluid an effective amount of a pharmaceutical formulation comprising at least one chemical compound, and at least one aqueous diluent, wherein the at least one chemical compound comprises a molecular weight of between about 400 MW and about 10,0000 MW, with protein binding of greater than 30% and greater than 70 Angstroms in cross sectional area; and
ameliorating and/or inhibiting brain cancer in the subject using the pharmaceutical formulation.

2. The method of claim 1, wherein the pharmaceutical formulation is administered for periods of longer than about 8 hours at a time.

3. The method of claim 1, wherein the pharmaceutical formulation is administered not less than every four weeks at least during the initial few months of administration.

4. The method of claim 1, further comprising solubilizing the at least one chemical compound in the at least one aqueous diluent.

5. The method of claim 1, further comprising solubilizing the at least one chemical compound in the at least one aqueous diluent using pegylation, liposomal encapsulation, emulsion carrying system, microgrinding into nano particles, or cyclodextrins.

6. The method of claim 1, wherein the at least one chemical compound comprises a pharmaceutically acceptable salt thereof.

7. The method of claim 1, wherein the at least one chemical compound comprises Irinotecan, SN-38, and/or a related derivative thereof.

8. The method of claim 1, wherein the at least one chemical compound comprises Irinotecan, wherein the method further comprises administering Irinotecan at about 5 to about 200 mgs per day for a period of administration of not fewer than 8 hours, and wherein the at least one aqueous diluent comprises 5% Dextrose or at 0.9% Sodium Chloride.

9. The method of claim 1, wherein the at least one chemical compound comprises Irinotecan, wherein the method further comprises administering Irinotecan such that SN-38 achieves levels above 18.0 pmol of SN-38 and possibly higher than SN-38 IC₅₀=10-750 nm (low end=U251, high end=U87) CPT-11 IC₅₀=10 uM-85 uM (low end=U251, high end=U87) will be sampled via an implantable CSF sampling device and via intermittent brain biopsies.

10. The method of claim 1, wherein the at least one chemical compound comprises Etirinotecan pegol, wherein the method further comprises administering Etirinotecan pegol at about 0.5 to 200 mgs per day for a period of administration of not fewer than 60 minutes, and wherein the at least one aqueous diluent comprises 5% Dextrose or at 0.9% Sodium Chloride

11. The method of claim 1, wherein the at least one chemical compound comprises Etirinotecan pegol, wherein the method further comprises administering Etirinotecan pegol such that SN-38 achieves levels above 18.0 pmol of SN-38 and possibly higher than SN-38 IC₅₀=10-750 nm (low end=U251, high end=U87) CPT-11 IC₅₀=10 uM-85 uM (low end=U251, high end=U87) will be sampled via an implantable CSF sampling device and via intermittent brain biopsies

12. The method of claim 1, wherein the at least one chemical compound comprises abraxane, Cabazitaxel, carfilozimb, docetaxel, doxorubicin, Etirinotecan pegol (NKTR-02), etoposide, NKTR-105, omacetaxine mepesuccinate, topotecan, paclitaxel, lapatinib, temsirolimus, or trametinib.

13. The method of claim 1, further comprising administering to the subject a pharmaceutical manufactured form of carboxylesterase inducing further conversion of CPT-11 to SN-38 and to expand the bioavailability of SN-38 to further treat the brain cancer.

14. The method of claim 1, further comprising administering to the subject a pharmaceutical manufactured form of atropine could be co-administered centrally to further tolerance of the medication.

15. The method of claim 1, further comprising adjusting a concentration of the at least one chemical compound based upon sampling of the subject's cerebrospinal fluid.

16. The method of claim 1, wherein the pharmaceutical formulation is administered to the subject via a treatment course which lasts at least two weeks and extends indefinitely.

17. The method of claim 1, wherein the brain cancer comprises metastatic cancer including small cell lung cancer, gastrointestinal cancer, breast cancer, testicular cancer, pancreatic cancer or primary brain tumors, wherein primary brain tumors comprise glioblastoma, anaplastic astrocytoma, or glioma.

18. The method of claim 1, further comprising administering the pharmaceutical formulation via a long catheter that is connected to either an implantable pump or an externalized pump for greater than 12 inches of catheter under the skin and preferably longer.

19. The method of claim 1, further comprising administering the pharmaceutical formulation using a kit including an implantable pump system including separately or together a ventricular catheter, an infusion catheter, a sterility packaging, patient identification card, infusion system identification card.

20. The method of claim 1, further comprising administering the pharmaceutical formulation to a thecal space of the subject depending on their toxicity profile.

21. A pharmaceutical formulation comprising at least one chemical compound and at least one aqueous diluent, for ameliorating and/or inhibiting brain cancer sensitive to cytotoxic effect, wherein the at least one chemical compound comprises a molecular weight of between about 400 MW and about 10,0000 MW, with protein binding of greater than 30% and greater than 70 Angstroms in cross sectional area.

22. A pharmaceutical formulation comprising at least one chemical compound for ameliorating and/or inhibiting brain cancer.
